(19) United States
(12) Patent Application Publication
(10) Pub. No.: US 2004/0215129 A1
(43) Pub. Date: Oct. 28, 2004

(54) Method and cycler for the administration of a peritoneal dialysis fluid

(75) Inventors: Raymond Anthony Edgson, Litlington (GB); Michael John Dunkley, Cambridge (GB); Richard J. Hammond, Cambridge (GB); Eric Wilkinson, Cambridgeshire (GB)

Correspondence Address: LERNER, DAVID, LITTENBERG,, KRUMHOLZ & MENTLIK, 600 SOUTH AVENUE WEST, WESTFIELD, NJ, 07090, US

(73) Assignee: Gambro AB, Lund (SE)

(21) Appl. No.: 10/746,670

(22) Filed: Dec. 22, 2003

Related U.S. Application Data
(62) Division of application No. 10/088,195, filed on Jul. 16, 2002.
Application No. 10/088,195 is a 371 of international application No. PCT/SE00/01772, filed on Sep. 14, 2000.

(30) Foreign Application Priority Data
Sep. 16, 1999 (SE) 9903331-8

(51) Int. Cl. A61M 1/00

(52) U.S. Cl. 604/29

(57) ABSTRACT

A method and apparatus for peritoneal dialysis are described The method includes draining a spent fluid from a patient into a second bag in a pressure chamber including a first bag for containing a fresh fluid for supply to a patient and a second bag for containing a spent fluid drained from a patient, by applying a reduced pressure to the second bag while weighing the first and second bags so as to control the draining of the spent fluid and supplying the fresh fluid to the first bag at a predetermined replenishment flow rate during draining of the spent fluid from the patient into the second bag independently of the influence of the reduced pressure. Apparatus for conducting such a method is also included.

### CROSS-REFERENCE TO RELATED APPLICATIONS

The present application is a divisional of co-pending U.S. patent application Ser. No. 10/088,195, filed Jul. 16, 2002, the disclosure of which is hereby incorporated by reference herein.

### FIELD OF THE INVENTION

The present invention relates to a method and cycler for the administration of a sterile medical fluid, such as a peritoneal dialysis fluid. More specifically, the present invention relates to a method and apparatus for operating a cycler for decreasing the cycle time.

### BACKGROUND OF THE INVENTION

Medical fluids intended for mammals, specifically for use in humans, are required to be sterile before being infused or applied to the mammal.

One known method for sterilizing a fluid is to heat the fluid to a sterilizing temperature and to hold the fluid at the sterilizing temperature during a sterilizing time period. To obtain a sterile medical fluid intended for infusion, the fluid is normally heated in an autoclave to about 121° C. for about 20 minutes to thereby produce the sterile medical fluid. After the sterilizing time has elapsed, the fluid should be cooled to a physiologically acceptable temperature before infusion.

Known methods and apparatus for sterilizing a fluid are disclosed, for example, in British Patent Nos. 1,450,030, 1,504,334, and 2,034,584, and U.S. Pat. No. 5,603,894. These prior art publications describe the preparation of a medical fluid starting from tap water and producing pure water by means of a reverse osmosis device, mixing a concentrate with the pure water to produce a non-sterile medical fluid, passing the non-sterile medical fluid through an on-line autoclave and delivering the sterile medical fluid to a recipient, such as a storage bag or a patient.

In the prior art, the complete medical fluid is first prepared in a non-sterile condition and then passes through an autoclave. If the medical fluid comprises heat sensitive components, these must not be exposed to too high a temperature. Normally, the temperature is increased up to the sterilizing temperature and the medical fluid is maintained at the sterilizing temperature for a sterilizing time period. If the temperature is 121° C., which is normal in an autoclave, the sterilizing time is 20 minutes to obtain a sterilizing dose F₀ of 20 minutes, (Bee discussion below for further details): Since the sterilizing effect is approximately exponential, an increase of the temperature by 10° C. means a lowering of the sterilizing time by ten times. If a sterilizing temperature of 131° C. is used, the sterilizing time should be 2 minutes, and if a sterilizing temperature of 141° C. is used, the sterilizing time should be 12 seconds, in order to obtain a sterilizing effect F₀ of 20minutes.

The fluid thus produced should be provided to a patient. For peritoneal dialysis, a cycler is normally used for introducing and removing the fluid to and from the patient. One such cycler is disclosed in International Application No. WO 95/20985 assigned to the applicant hereof, the content of which is incorporated herein by reference thereto.

The cycler according to this publication is provided with a pressure chamber enclosing two tags; namely, a heater bag and a drain bag. The bags may be arranged as a double bag. The bags are arranged on a weighing device, such as a pair of scales. The weighing device controls the weight of the combined bags, and valves control the flow of fluid into and out of these bags in order to perform a patient drain and a patient fill. Moreover, the cycler replenishes the heater bag from a supply of fresh fluid and empties the drain bag to a waste receivers.

According to the present invention, the fresh fluid may be provided from an autoclave which is typically only operated at a constant flow rate, which should be as low as possible for reducing the requirement of heat transfer during the autoclave cycle. Thus, the replenishment fluid flow rate is relatively low.

However, the efficiency of the peritoneal dialysis is dependent on, inter alia, the number of exchanges of fluid during a treatment period, such as a night. Thus, in some case, the low replenishment fluid flow rate may limit the efficiency of the treatment.

Thus, there is a need for a method of operating a cycler of the type mentioned above, in which the replenishment time may not be a hindrance for the efficient treatment. This is more important when the cycler is directly connected to an on-line autoclave, having a reduced replenishment fluid flow rate which moreover should be constant.

Accordingly, one object of the present invention is to provide a method and a cycler in which the cycle time is reduced, in spite of a limited replenishment fluid flow rate.

### SUMMARY OF THE INVENTION

In accordance with the preset invention, this and other objects have now been realized by the invention of a method of peritoneal dialysis utilizing a pressure chamber including a first bag including an inlet for containing a fresh fluid for supply to a patient and a second bag for containing a spent fluid drained from a patient, and weighing means for weighing the first and second bags, the method comprising draining the spent fluid from the patient into the second bag by applying a reduced pressure to the second bag while weighing the first and second bags by the weighing means so as to control the draining of the spent fluid, and supplying the fresh fluid to the first bag at a predetermined replacement flow rate during the draining of the spent fluid from the patient into the second bag independently of the influence of the reduced pressure. Preferably, the method includes applying the reduced pressure to both the first and second bags during the draining of the spent fluid from the patient into the second bag.

In accordance with one embodiment of the method of the present invention, the method includes supplying the fresh fluid to the first bag at the predetermined replenishment flow rare by means of a volumetric pump provided at the inlet of the first bag.

In accordance with another embodiment of the method of the present invention, the predetermined replenishment flow rate comprises a constant flow rate. Preferably, the method includes supplying the fresh fluid to the first bag at the constant flow rate until a predetermined replacement volume of the fresh fluid has been supplied to the first bag.

In accordance with another embodiment of the method of the present invention, the method includes controlling the draining of the spent fluid from the patient into the second bag by means of the weighing means by correcting the amount of the spent fluid drained from the patient by the amount of the fresh fluid supplied to the first bag. Preferably, the method includes terminating the draining of the spent fluid from the patient into the second bag when a predetermined volume of spent fluid has been drained into the second bag. In another embodiment, the method includes terminating the draining of the spent fluid from the patient into the second bag when a predetermined time period has elapsed from the start of the draining of the spent fluid from the patient into the second bag. In yet another embodiment, the method includes terminating the draining of the spent fluid from the patient into the second bag when the rate of flow of the spent fluid into the second bag is below a predetermined flow rate, and including determining the rate of flow of the spent fluid into the second bag by means of the weighing means.

In accordance with another embodiment of the method of the present invention, the method includes filling the patient with the fresh fluid from the first bag and emptying the spent fluid from the second bag to a waste receiver by providing a positive pressure to the second bag, wherein the supplying of the fresh fluid to the first bag is carried out during the draining of the spent fluid from the patient into the second bag by pumping the fresh fluid at a predetermined flow rate independent of the influence of the negative pressure on the first bag. In a preferred embodiment, the method includes initiating the supply of the fresh fluid to the first bag during the emptying of the spent fluid from the second bag, and initiating the draining of the spent fluid from the patient after termination of the emptying of the spent fluid from the second bag.

In accordance with another embodiment of the method of the present invention, the filling of the patient with the fresh fluid is carried out under a positive pressure in the pressure chamber, the emptying of the spent fluid from the second bag is carried out under a positive pressure in the pressure chamber, and the supply of the fresh fluid to the first bag is carried out during either the emptying of the spent fluid from the second bag or the draining of the spent fluid under the control of a positive displacement pump irrespective of the pressure in the pressure chamber.

In accordance with another embodiment of the method of the present invention, the method, includes filling the patient with the fresh fluid from the first bag and emptying the spent fluid from the second bag to a waste receiver by providing an increased pressure to the second bag, wherein the supply of the fresh fluid to the first bag and the draining of the spent fluid from the patient are carried out at least partially simultaneously. In a preferred embodiment, the method includes initiating the supplying of the fresh fluid to the first bag after termination of the filling of the patient with the fresh fluid from the first bag, and continuing the supplying of the fresh fluid to the first bag during the draining of the spent fluid from the patient in the second bag. Preferably, the method includes continuing the supplying of the fresh fluid to the first bag during the emptying of the spent fluid from the second bag to the waste receiver.

In accordance with another embodiment of the method of the present invention, the method includes heating the fresh fluid to a temperature of about 37° C. during the supplying of the fresh fluid to the first bag. In a preferred embodiment, the method includes terminating the supplying of the fresh fluid to the first bag and initiating the filling of the patient with the fresh fluid from the first bag when the temperature of the fresh fluid in the first bag reaches about 37° C.

In accordance with the present invention, apparatus has also been provided for peritoneal dialysis comprising a pressure chamber including a first bag including an inlet for retaining a fresh fluid for supply to a patient and a second bag for retaining a spent fluid drained from a patient, weighing means for weighing the first and second bags, draining means for draining the spent fluid into the second bag by applying a negative pressure to the second bag in the pressure chamber under the control of the weighing means, and supply means for supplying the fresh fluid to the first bag, the supply means including a pump for pumping the fresh fluid at a predetermined replenishment flow rate during the draining of the spent fluid into the second bag irrespective of the negative pressure applied to the second bag. In a preferred embodiment, the draining means comprises pressure means for supplying a negative pressure in the pressure chamber.

In accordance with one embodiment of the apparatus of the present invention, the supply means comprises a volumetric pump disposed at the inlet of the first bag. Preferably, the volumetric pump is adapted to pump the fresh fluid into the first bag at a constant replenishment flow rate. In a preferred embodiment, the volumetric pump is adapted to pump the fresh fluid into the first bag until a predetermined volume has been supplied to the first bag.

In accordance with another embodiment of the apparatus of the present invention, the weighing means in adapted to control the draining of tho spent fluid into the second bag corrected by the supplying of the fresh fluid to the first bag by the volumetric pump. In a preferred embodiment, the apparatus includes interruption means for interrupting the draining of the spent fluid into the second bag when a predetermined volume of the spent fluid has been drained into the second bag. In another embodiment, the apparatus includes interruption means for interrupting the draining of the spent fluid into the second bag when a predetermined time has elapsed from the initiation of the draining of the spent fluid into the second bag. In yet another embodiment, the apparatus includes interruption means for interrupting the draining of the second fluid into the second bag when the inlet flow rate of the spent fluid into the second bag is less than a predetermined inlet flow rate, the inlet flow rate determined by the weighing means.

In accordance with another embodiment of the apparatus of the present invention, the apparatus includes the weighing means adapted to determine the inlet flow rate of the spent fluid into the second bag based on the change of weight of the combined first and second bags.

In accordance with another embodiment of the apparatus of the present invention, the apparatus includes filling means for filling the first bag with the fresh fluid and emptying means for emptying the spent fluid from the second bag to a waste receiver, whereby the supplying of the fresh fluid to the first bag and the draining of the spent fluid from the patient into the second bag are carried out at least partially simultaneously. In a preferred embodiment, the apparatus includes means for initiating the supplying of the fresh fluid to the first bag during the emptying of the spent fluid from the second bag and for initiating the draining of the spent fluid from the patient into the second bag after terminating the emptying of the spent fluid from the second bag.

In accordance with another embodiment of the apparatus of the present invention, the apparatus includes pressure control means for controlling the pressure in the chamber whereby the draining of the spent fluid from the patient into the second bag is carried out under a negative pressure, the filling of the patient with the fresh fluid from the first bag is carried out under a positive pressure, the emptying of the spent fluid from the second bag to the waste receiver is carried out under a positive pressure, and the supplying of the fresh fluid to the first bag is carried out during either the emptying of the spent fluid from the second bag or the draining of the spent fluid from the patient into the second bag under control of a positive displacement pump irrespective of the pressure in the pressure chamber.

In accordance with another embodiment of the apparatus of the present invention, the apparatus includes means for initiating the supplying of the fresh fluid to the first bag after termination of the filling of the patient with the fresh fluid and for continuing the supplying of the fresh fluid to the first bag during the draining of the spent fluid from the patient. In a preferred embodiment, the including means includes means for continuing the supplying of the fresh fluid to the first bag during the emptying of the spent fluid from the second bag.

In accordance with another embodiment of the apparatus of the present invention, the apparatus includes heating means for heating the fresh fluid in the first bag to a temperature of about 37° C. In a preferred embodiment, the apparatus includes means for terminating the supplying of the fresh fluid to the first bag and for initiating the filling of the patient with the fresh fluid from the first bag when the temperature reaches about 37° C.

In accordance with another embodiment of the apparatus of the present invention, the apparatus includes valve means for controlling the fluid flow to and from the first and second bags in a preferred embodiment, the valve means comprises a first valve for controlling the flow of the fresh fluid into the first bag, a second valve for controlling the flow of the fresh fluid from the first bag to the patient, a third valve for controlling the flow of the spent fluid into the second bag, and a fourth valve for controlling the flow of the spent fluid out of the second bag. Preferably, the apparatus includes valve control means for opening the first valve only when the second valve is closed. In another embodiment, the apparatus includes valve control means for opening the third valve only when the second valve and the fourth valve are closed. In another embodiment, the apparatus includes valve control means for opening the third valve only when the fourth valve is closed, and for opening the fourth valve only when the third valve is closed.

In accordance with another embodiment of the apparatus of the present invention, the apparatus includes pressure control means for controlling the pressure in the pressure chamber whereby a positive pressure is maintained in the pressure chamber when the second valve and the fourth valve are opened and a negative pressure is maintained in the pressure chamber when the third valve is opened, and either a positive or negative pressure is maintained in the pressure chamber when the first valve is opened.

In accordance with another embodiment of the apparatus of the present invention, the apparatus comprises an integrated double bag including both the first bag and the second bag.

In accordance with another embodiment of the apparatus of the present invention, the supply means comprises a pump and a flow meter for measuring the replenishment fluid flow rate.

In accordance with the present invention, there is thus provided a method of operating a cycler and a cycler intended for peritoneal dialysis, comprising a pressure chamber provided with a first bag for enclosing a fresh fluid intended to fill a patient and a second bag for enclosing a spent fluid to be drained from a patient, the first and second bag being arranged at a weighing device for weighing the combined weight thereof.

In order to reduce the total cycle time, the cycler comprises a draining device for draining the spent fluid into the second bag supervised by the weighing device, and a replenishment device for replenishing the first bag at a predetermined replenishment fluid flow rate during the draining step. Preferably, the draining device is a pressure device for generating an underpressure in the pressure chamber comprising the bags during the draining step. Moreover, the replenishment device may be a volumetric pump arranged at the inlet of the first bag.

The volumetric pump may be arranged to pump the fluid into the first bag at a constant fluid flow rate, whereby the cycler and autoclave is easier to control. The volumetric pump is arranged to replenish the first bag at a constant fluid flow rate until a predetermined replenishment volume has been introduced into the first bag.

The weighing device is arranged to control the draining step corrected for by the replenishment of the first bag.

The cycler may be arranged to interrupt the draining step when a predetermined volume has been drained into the second bag, or when a predetermined time has elapsed from the start of the draining step. Alternatively, the cycler may be arranged to interrupt the draining step when an inlet flow rate into the second bag is below a predetermined flow rate, the inlet flow rate being determined by the weighing device.

The cycler according to the, present invention is operated in four phases, in the following order: a drain phase for draining spent dialysate from a patient connected to the second bag; a fill phase for filling the patient with fresh fluid from the first bag; an emptying phase for emptying the spent dialysate in the second bag to a waste receiver; and a replenishment phase for replenishing the first bag with fresh fluid. The replenishment phase and the drain phase takes place at least partially simultaneously. The cycler may be arranged to initiate the replenishment phase during the emptying phase and to initiate the drain phase after the termination of the emptying phase.

In an alternative embodiment the cycler may be operated in the four phases in the following order: a drain phase for draining spent dialysate from a patient connected to the second bag; an emptying phase far emptying the spent dialysate in the second bag to a waste receiver; a fill phase for filling the patient with fresh fluid from the first bag; and a replenishment phase for replenishing the first bag with fresh fluid. Also in this case, the replenishment phase and the drain phase takes place at least partially simultaneously.

In order that the fluid intended to be introduced into a patient is delivered at a temperature close to body temperature, a heating device is arranged to expose the first bag to heat energy, during the replenishment phase, for heating the fluid in the first bag to a temperature close to 37 degrees Celsius. The cycler is arranged to terminate the replenishment phase and to initiate the fill phase only when the temperature of the fluid in the first bag is close to 37 degrees Celsius.

The cycler may be provided with valves for controlling the fluid flow to and from the first bag and the second bag. There is arranged a first valve for controlling the fluid flow into the first bag from the replenishment device, a second valve for controlling the fluid flow out from the first bag to a patient line, a third valve for controlling the fluid flow into the second bag from the patient line and a fourth valve for controlling the fluid flow out from the second bag. The first valve is opened only when the second valve is closed and vice versa. The third valve is opened only when the second valve and the fourth valve are closed.

The pressure apparatus is arranged to expose the pressure chamber to a positive pressure when the second valve is opened and when the fourth valve is opened, and a negative pressure when the third valve is opened, and either a positive or negative pressure when the first valve is opened.

As an alternative to the volumetric pump, there may be arranged any type of pump, supplemented with a flow meter, which measures the replenishment fluid flow rate of the fluid provided to the first bag. In this manner, the weighing device can be corrected for the replenishment fluid flow rate and thereby obtain full control of the drain fluid flow rate as well as the fill fluid flow rate.

### BRIEF DESCRIPTION OF THE DRAWINGS

Further objects, advantages and features of the present invention will appear from the following detailed description of several embodiments shown on the drawings, in which:

FIG. 1 is a top, elevational, schematic view of a first embodiment of apparatus for sterilizing a heat sensitive fluid intended to be used according to the present invention;

FIG. 2 is a top, elevational, schematic view similar to FIG. 1 of a second embodiment of apparatus according to the present invention;

FIG. 3 is a top, elevational, schematic view similar to FIG. 2 of a portion of a third embodiment of apparatus according to the present invention;

FIG. 4 is a top, elevational, schematic view similar to FIG. 1 of a third embodiment of apparatus according to the present invention;

FIG. 5 is a side, elevational, schematic view of a first embodiment of a cycler which may be connected to the apparatus according to FIG. 2, 3 or 4;

FIG. 6 is a graphical representation of a time diagram of the fluid flows in the cycler according to FIG. 5;

FIG. 7 is a graphical representation of an alternative time diagram similar to FIG. 6; and

FIG. 8 is a side, elevational, schematic view similar to FIG. 5 of a second embodiment of the cycler shown therein.

### DETAILED DESCRIPTION

The fluid to be sterilized comprises a first non-heat-sensitive portion and a second heat sensitive portion. The two portions may be delivered separately to the sterilizing device into two separate inlets, 1 and 2.

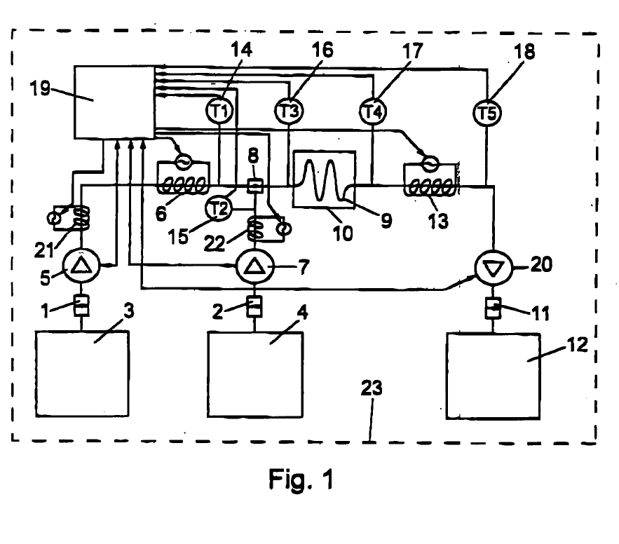

With reference to FIG. 1, the first non-heat-sensitive component, which may comprise sodium chloride dissolved in water, is enclosed in a vessel 3 connected to the inlet 1. The second heat sensitive component, which may comprise glucose, to enclosed in a vessel 4 connected to the inlet 2. The fluid components are preferably provided at a temperature at which each component is relatively stable, such as room temperature.

The first fluid portion from vessel 3 provided to inlet 1 is impelled by a first pump 5 to a heater 6, in which the first fluid portion is heated to a first high temperature. The second fluid portion is impelled by a second pump 7 and mixed with the first fluid portion at a mixing point a arranged downstream of the heater 6. During the mixing, the second fluid portion is rapidly heated to a sterilizing temperature, while the first fluid portion is cooled to the same sterilizing temperature. The second fluid portion does not make direct contact with the heater surface, and damage is therefore minimized.

In order to promote rapid mixing, the fluids are impelled at such conditions that turbulent flow prevails at least after the mixing point 8. In addition, flow mixing means may be arranged in the flow path, such as at the mixing point 8 or in the flow path downstream of mixing point 8. Such flow mixing means may be flanges Nor wings in the flow path.

The mixed fluid portions pass through a sterilizing tube section 9 dimensioned to provide a predetermined resident or sterilizing time for the mixed fluids at the sterilizing temperature. The tube section may be insulated as indicated by box 10 to maintain the mixed fluids at the sterilizing temperature for the sterilizing time. After the sterilizing time, the mixed fluids are sterile, since the second fluid portion has been subjected to the sterilizing temperature during a sterilizing time and the first fluid portion has been exposed to a still higher temperature and still longer time, thus being oversterilized.

The sterilizing dose is a function of temperature and time and is defined according to the formula:

$${\int }_{0}^{t} {F}_{0} = {\vert}_{10}^{( T - 121 ) / 10} ⅆ t$$

in which

F₀=the sterilization dose in minutes

T=temperature

t=time

If the sterilizing temperature is 121° C. and the time is 20 minutes, a sterilization dose of 20 minutes is obtained. If the sterilizing temperature is 141° C. and the time is 12 seconds, a sterilization dose F₀ of 20 minutes is also obtained. A sterilizing dose F₀ of 20 minutes is normally considered sufficient. However, in certain applications, a sterilizing dose F₀ of 10 minutes or even lower ray be sufficient.

In the above example, the first fluid portion may comprise sodium chloride at a concentration of about 150 mM, sodium lactate at a concentration of about 38.8 mM, magnesium chloride at a concentration of about 0.56 mM and calcium chloride at a concentration of about 1.89 mM. The second fluid portion may comprise glucose at a concentration of about 40%; i.e., 400 g glucose per liters of solution. The first fluid portion flow rate is about 45 ml/min and the second fluid portion flow rate is 5 ml/min. The resulting mixture has the following composition: sodium chloride 135 mM, sodium lactate 35 mM, magnesium chloride 0.5 m, calcium chloride 1.7 mM and glucose 4%. The first fluid portion is heated from about 20° C. to 155° C. by the heater 6. The second fluid portion is heated from about 20° C. to 141° C. during mixing, while the first fluid portion is cooled from about 155° C. to 141° C. The resident or sterilizing time is about 12 seconds, resulting in a sterilizing dose F₀ of 20 minutes. The resulting sterilized fluid mixture is cooled by a cooler 13 and delivered to an outlet 11 and collected in a vessel 12. A pump 20 or other device may be arranged to control the flow to the vessel 12. The sterile fluid may be used as a peritoneal dialysis solution to be delivered to the peritoneal cavity of a patient.

Other medical fluids may be produced by the device according to the invention, such as hemodialysis solutions, infusion solutions used in hemodiafiltration or hemofiltration, replacement fluids for infusion in the blood, wound irrigation solutions, rinsing solutions, etc. Moreover, nutritional solutions often comprises amino acids, which are heat sensitive, and glucose, which is heat sensitive, and cannot be sterilized together with amino acids. Certain drugs, such as insulin, may be produced or included in a fluid administered to a patient, and the drug component may be heat sensitive. Certain medical fluids comprise peptides, proteins or fragments thereof, which normally are heat sensitive. Preservation fluids for blood component handling may also comprise heat sensitive components, at least glucose. In certain cases, glucose is replaced with or complemented with glucose polymers, di-saccharides, tri-saccharides, etc. certain carboxylic acids are heat sensitive and may be included in such fluids. Solutions comprising calcium or magnesium ions and carbonate or bicarbonate ions may precipitate upon exposure to a sterilizing temperature, and need to be sterilized with the carbonate or bicarbonate separate from the calcium or magnesium containing solution.

In order to control the above procedure, one or several temperature sensors are provided. A first temperature sensor 14 may be arranged immediately drownstream of the beater 6 to determine the temperature of the first fluid portion after heating. A second temperature sensor 15 may be arranged between the second inlet 2 and the mixing point 8 to determine the temperature of the second fluid before mixing. A third temperature sensor 16 may be arranged downstream of the mixing point to determine the mixing temperature. A fourth temperature sensor may be arranged downstream of the sterilizing section 9 to determine the sterilizing temperature. A fifth temperature sensor 18 may be arranged downstream of cooler 13 to determine the temperature of the fluid delivered to vessel 12. Not all of theme five temperature sensors are needed, so that one or more thereof may be excluded.

A control processor 19 may be arranged to control the sterilizing device according to the present invention. As shown in FIG. 1, the five temperature sensors are connected to the processor as well as the pumps 5, 7 and 20 to provide measurements of the temperatures and flow rates. The pumps 5, 7 and 20 may be volumetric pumps also acting as flow meters. Alternatively, separate flow meters may be provided. The processor controls the heater 6 to provide the required temperature downstream of the heater, as measured by temperature sensor 14, to provide the sterilizing temperature after mixing as measured by temperature sensors 16 and 17. The processor calculates the residence time in the sterilizing section 9 based on the flow rates of pumps 5 and 7 and the known volume of the sterilizing section 9. Finally, the processor may determine the obtained sterilizing dose F₀.

The control processor 19 may, obtain all necessary information in order to calculate the sterilizing effect from the flow rates of pumps, 5 and 7, and the temperature of sensor 17.

As also shown in FIG. 1, the fluids provided to inlets 1 and 2 may be preheated by preheaters 21 and/or 22.

Since the sterilizing apparatus shown in FIG. 1 is intended to heat the fluids to temperatures well above 100° C., it is required to keep the fluids from boiling. This may be done by enclosing the entire apparatus in an enclosure 23, as shown by broken lines in FIG. 1, and raising the pressure inside the enclosure to a pressure sufficient to prevent boiling, such as 3-6 Bar absolute pressure. Another method would be to arrange a high pressure zone in the pipes or lines between the pumps, 5, 7 and 20.

It is known that glucose decomposes when exposed to heat, and is thus a heat sensitive component of the fluid. Glucose also decomposes during storages. It is known that several factors influence the decomposition of glucose, among which are pM, temperature, time, glucose concentration and mixing with certain ionic components. Glucose decomposes into components, some of which may be more or legs toxic or are able to induce toxic reactions by including precursors for such reactions. If the resulting fluid is to be used as a medical fluid for infusion into a human being or other mammal, the toxic components or precursors should be minimized.

In order to sterilize the fluid it is necessary to expose the fluid to sterilizing conditions. There are several methods available, such as heat sterilization (autoclaving), filter sterilization and other methods. The present invention is limited to heat sterilization.

During heat sterilization, it is known that decomposition of glucose can be minimized if glucose is sterilized during a short time at a high temperature. The rationale is that the decomposition reaction is less sensitive to high temperature than the sterilizing reaction.

In order to minimize decomposition before sterilization, it is advantageous to store the fluid at a low pH and at a high concentration, which is suggested according to the present invention. The pH may be from about 2.6 to 5.0, and preferably pH =3.2. The concentration may be above 15% or above 20% with 40% to 50% being, preferred, calculated as weight of glucose per liter of solution.

The sterilization may take place during a short time and at a pH of below about 5.5 and at a dilution concentration. It is believed that the short time is of greater importance than the other factors for avoiding decomposition into toxic components of glucose during the sterilization process.

It is also recognized that glucose may decompose into precursors for AGE, advanced glucosylation end products. When a glucose solution comprising precursors for AGE contacts proteins in the body, a non-enzymtic reaction takes place resulting in ACE formation. The long term effect of AGE is still not well known. Gentle heat sterilization of glucose as suggested in the present invention is expected to reduce the level of glucose degradation products of the type of AGE precursors.

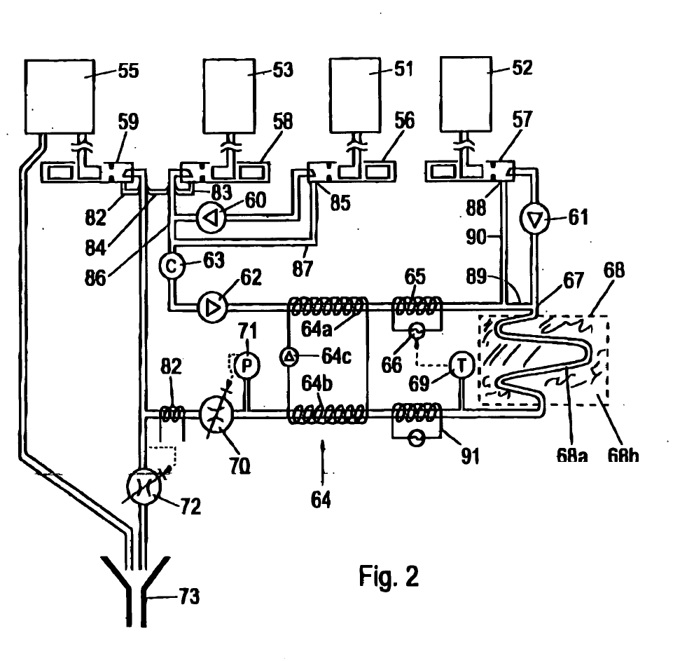

An alternative embodiment of the present invention is shown in FIG. 2. In this embodiment, the sterilizing device according to the present invention is integrated with a PD monitor arranged to provide a PD solution to a patient. The PD solution is prepared from two concentrates provided in two concentrate bags, 51 and 52, and connected to concentrate input connectors, 56 and 57, and a supply of pure water, for example provided from a reverse osmosis RO-unit 53 connected to a water input connector 59 for connection to a potable water supply. The sterilized PD fluid is delivered to a PD cycler 55, which is, in turn, connected to a PD fluid output connector 59 for delivery to the patient.

Each of the three input connectors and the output connector may be arranged as a heat sterilizable connector Such a heat sterilizable connector device is described in International Application No. WO 96/05883, which is incorporated herein by reference thereto.

Each of the inputs, 56, 57 and 58, and the output 59 is arranged as a connector device. Input 56 is arranged to connect a first concentrate bag 51 to a first metering pump 60 and input 57 is arranged to connect a second concentrate bag 52 to a second metering pump 61. Input 55 is connected to RO-unit 53 and a third pump 62 is arranged to pump pure water from RO-unit 53.

Pumps 62 and 60 are driven to mix the concentrate from bag 51 with pure water from RO-unit 53 to provide a desired concentration. A conductivity cell 63 may be arranged to measure the conductivity of the mixture and may control the pump 60 and/or 62 to obtain the required conductivity and thus the desired concentration. Pump 62 is preferably driven to provide a constant flow of, for example, 54 ml/min and at the same time increase the pressure to about 3 to 6 Bar absolute pressure to avoid boiling during sterilization. The fluid provided so far to the first heat-insensitive fluid mentioned above.

The first fluid passes through a first heat exchanger 64 comprising a primary circuit 64a for heating the first fluid, for example from 20° C. to 100° C. Then, the first fluid passes through a heater 65 such as an electric heater powered by an electric power supply 66 to heat the first fluid to a temperature of 155° C.

The second, heat sensitive, fluid from bag 52 is pumped by pump 61, at a flow rate of 6 ml/min to a mixing point 67 immediately downstream of heater 65 to mix with the first fluid. The second fluid is thus rapidly heated from room temperature to a temperature of about 141° C. by being mixed with the hot first fluid, which at the same time cools down to about 141° C.

Then, the mixed fluids pass through a sterilizing unit 68 comprising a tube 68a of a length suitable for providing a residence time giving the required sterilizing time, such as 12 seconds. The tube is embedded in an insulating material 68b to minimize the temperature decrease during the residence time.

Immediately downstream of the sterilizing unit 68 is a temperature sensor 69, which controls the power supply 66 so that the temperature is the desired sterilizing temperature, such as 141° C.

Pump 61 is controlled to deliver the heat sensitive fluid in the amount desired. For example, if the heat sensitive fluid is glucose at a concentration of 40%, the flow rate should be 6 ml/min to give a final concentration of 4% if the first flow rate is 54 ml/min. If a concentration of 1.5% is desired, the flow rate should be 2.1 ml/min and if a concentration of 2.5% should be obtained, the flow rate should be 3.6 ml/min. In each case, the temperature sensor adjusts the power supply to heat the first fluid to a suitable temperature so that the sterilizing temperature is obtained.

After the sterilizing unit 88, the now sterilized fluid enters the secondary circuit 64b of the heat exchanger 64 to rapidly decrease the temperature of the sterilized fluid, for example to 60° C. Then, the sterilized fluid passes a flow restrictor 70 to decrease the pressure to close to atmospheric pressure. Preferably, the flow restrictor 70 is controlled by a pressure sensor 71, so that the pressure before the restrictor is the desired pressure to prevent boiling, such as 6 Bar absolute pressure.

From the flow restrictor 70, the sterilized fluid is delivered to the output 59, which is connected to a PD cycler 55. A pressure relief valve 72 is arranged to connect the sterilized fluid to a waste receiver 73 if the pressure of the fluid exceeds a predetermined value, such as 150 mmHg above atmospheric pressure.

The PD cycler may be of the type described in International Application No. WO 95/20985, comprising a pressure chamber. A disposable line set is connected between the outlet connector and the patient and comprises a heater bag and a drain bag, a drain line and a supply line. The heater bag and a drain bag are arranged on a weighing device, such as a pair of scales. Four valves in a valve unit are arranged to operate on the drain and supply lines Finally, the line set comprises a PD patient connector for connection to a catheter ending in the peritoneal cavity of the patient. The PD replenishment fluid from outlet 159 is supplied to the heater bag by means of the valve unit until the scales indicate that the heater bag has been replenished to a predetermined volume, such as 3 liters. Then the patient is drained by exposing the pressure chamber to a subpressure to withdraw fluid in the peritoneal cavity of the patient out through the open valve unit into the drain bag. The combined weight of heater bag and drain bag is weighed and the drain phase is terminated when it is determined that the drain flow rate is below a predetermined limit or a drain time has elapsed. The drain flow rate is determined by means of the weighing device. Then, the pressure chamber is exposed to an overpressure and the valve unit is opened to allow the replenished and sterilized PD fluid to flow into the peritoneal cavity of the patient. The flow rate and the delivered fluid volume are monitored and the fill phase is terminated when a desired fill volume has been delivered. The temperature of the heater bag is controlled by a heating device and temperature sensor so that the fluid delivered has a temperature of about 37° C. Finally, the drain bag is emptied to a waste receiver by opening the valve unit and exposing the pressure chamber to an overpressure.

When the patient has been exposed to a fluid exchange as described above, the PD fluid is left in the peritoneal cavity for a dwell time until the next exchange cycle. During the dwell, the sterilizing device provides new replenishment sterile fluid to the heater bag. It takes about 33 minutes to produce a volume of 2 liters if sterile fluid is produced at 60 ml/min.

It may be desirable to include; a cooler 82 after the flow restrictor 70 to further decrease the temperature before delivering the fluid to the heater bag. The cooler may be a Peltier cooler or a heat exchanger of conventional design, using cold water or a cooling medium as heat energy absorption medium. A cooler 91, such as a Peltier cooler, may alternatively or additionally be placed after residence device 68 and before heat exchanger 64, in order to rapidly cool the heat sensitive mixture to a safe temperature, such as from 141° C. to 120° C. In this way, the heat sensitive component is heated rapidly from room temperature to sterilization temperature of 141° C. at mixing point 67, is maintained at the sterilizing temperature during 12 seconds by residence device 68 and is then rapidly cooled to 120° C. by Peltier cooler 91 and then further cooled to room temperature in the slightly slower heat exchanger 64.

The sterilizing device needs to be disinfected at suitable intervals, for example once per day or once per week. For that purpose, the side openings of the connector devices, 56, 57, 58 and 59, are used. The side opening 83 of RO inlet 58 is connected to the side opening 82 of outlet 59 through a line 84. The side opening 85 of first inlet 56 is connected to the flow line 86 between RO inlet 59 and the pump 62 through a line 87. The side opening as of second inlet 57 is connected to the line 89 between heater 65 and sterilizing unit 68 through a line 90.

During disinfection, the sterilizing device is filled with pure water obtained from the RO-unit. Then, connectors 57, 55 and 59, are disconnected from the respective sources.

Thus, the RO-inlet connector 58 and the outlet connector 59 are connected through line 84 and side openings 82 and 83. The second inlet connector 57 is in the same position 80 that a circulating path is obtained through pump 61, line segment 89, line 90, side opening 88 and inlet 57. A disinfecting solution is provided in a vessel connected to the first inlet 56. The disinfecting fluid may be sodium carbonate, citric acid or any other known disinfection fluid. Pumps 62 and 61 are operated to circulate the fluid in the circuits. Finally, pump 60 is operated to infuse disinfection fluid into the water until a sufficient disinfectant concentration has been obtained. The surplus water is rejected through relief valve 72 to the waste receiver 73. Pump 62 circulates the disinfection fluid through the complete sterilization device and the outlet 59 is connected to the inlet 59 through line 84 to complete the circuit. The disinfection fluid may be left in the machine until the next use. Before the next use, the machine is rinsed with pure water through inlet 58 from the source of RO-water.

Descaling with citric acid or other descaling agent is performed in the same manner.

In order to avoid dripping from the connectors, the inlet connectors, 56, 57 and 58, and the outlet connector 59 are positioned at the highest position of the flow path and at the same level.

The machine may be emptied by opening all inlets, 56, 57 and 58, and the outlet 59 and by , opening the relief valve 72, which is positioned at the lowest point of the flow path, and allowing air to enter all lined and devices.

During chemical disinfection and/or descaling, the heater 65 may be turned off or adjusted to heat the fluid Lo a low temperature. The flow restrictor 70 may be opened.

In heat sterilization, the fluid in the entire circuit is heated to 121° C. and circulated for at least 20 minutes to obtain sterilization of the entire circuit. In this case, pressure relief valve 72 is operated to permit a pressure of 2 Bar, thereby preventing boiling of the water in the circuit at 121° C.

The same or a similar procedure may be used for sterilizing the flow path of the sterilizing device. The fluid circuit is arranged for a treatment with all connectors inserted in respective bore in the non-engaged position. The circuit is filled with water, which is circulated by pump 62. Flow restrictor 70 is opened and relief valve 72 is adjusted to a pressure of from about 2 to 3 Bar absolute pressure. First inlet connector 56 is operated to connect the vessel 51 to the circuit. Then, pump 60 is operated to introduce some fluid (electrolyte fluid) in the circuit until the pressure reaches about 2 to 3 Bar absolute pressure. Since the fluid circuit is relatively non-compliant, the volume of fluid introduced is small. Then, the heater is activated to heat the water present in the circuit to a temperature of about 121° C. and the circulation continues for 20 minutes or longer, until sterilization is obtained. Pump 61 is operated simultaneously to sterilize the circuit comprising inlet connector 57.

After sterilization has been obtained, RO inlet 58 is activated to connect RO-unit 53 to the circuit and at the same time disconnect bypass line 84. Pump 60 is stopped, and heater 65 is activated. Flow restrictor 70 is activated and pressure relief valve 72 is adjusted to the normal value of 150 mmHg overpressure. Thus, sterile water is produced and delivered to the waste 73 through relief valve 72. Then, the second inlet is activated to connect vessel 52 and pumps 60 and 61 are operated to provide a PD fluid. When stable conditions are obtained, the outlet 59 is activated to deliver sterilized fluid to the heater bag.

In some cases, the heat sensitive component may be introduced together with the remaining components, and the bag 52, connector 57 and the corresponding pump 61 can be dispensed with. Instead, the other components of the fluid may be entered in the same way as component 51, i.e., mixed with water and the remaining components before heat sterilization.

During the drain and fill phases of the PD cycler, the sterilizing device may continue to produce PD fluid. However, since the valve unit is closed, the PD fluid produced is directed to the waste receiver 73 through relief valve 72. Since the drain and fill phases may last up to 20 minutes or more, a considerable amount of PD fluid is wasted. To minimize such waste, pumps 60 and 61 maybe stopped during the periods when the heater bag is not being filled, and the sterilizing device is only producing and wasting sterile water.

The first and/or second concentrates may comprise the same substances or components as mentioned above, however, with the contents of the first vessel 51 concentrated by omitting some of the water. The contents of the first vessel may be concentrated for example 30 to 40 times.

In an alternative embodiment, the PD fluid is intended to comprise bicarbonate instead of or in addition to lactate. Calcium cannot be included in the same vessel as bicarbonate, because of the risk of precipitation of calcium carbonate. In that case, the calcium chloride may be included in the second vessel 52 in a suitable concentration. The calcium concentration will then be proportional to the glucose concentration, which may result in a calcium neutral PD fluid. Another advantage of including the calcium ions in the second vessel is that scaling of the pipe system is avoided before the mixing point 67, and the requirement for descaling would decrease.

Further components may be included in the fluid flow before pump 62, by the inclusion of a further bag 51a, connector 56a and pump 60a, in parallel with vessel 51.

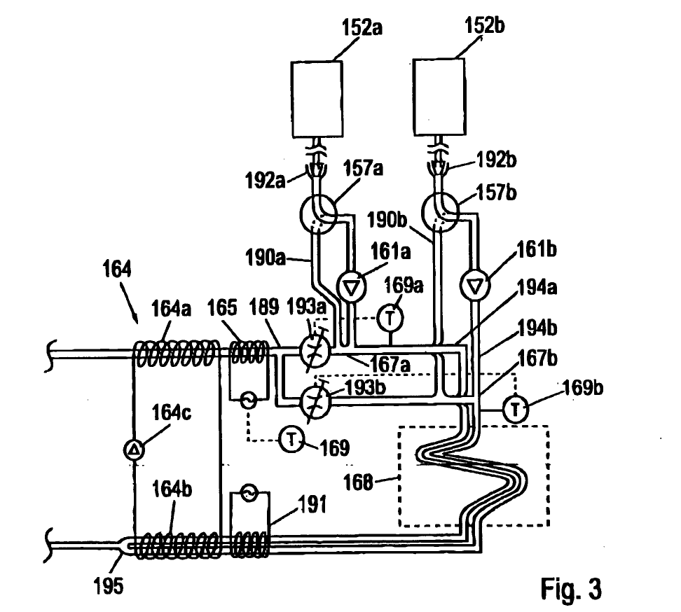

Each of the sterilizable connectors may be replaced by a conventional connector device and a three way valve of conventional type, as shown in more detail in FIG. 3, which shows an alternative embodiment of the present invention.

FIG. 3 shows an alternative design of a mixing system delivering the mixed fluids in parallel through the residence device. FIG. 3 shows only the right-hand portion of FIG. 2 to the right of pump 62 and pressure sensor 70. The left-hand portion may be identical to the embodiment of FIG. 2. The same components as in FIG. 2 have received the same reference numerals, but adding 100 to the reference numbers. Thus, there is shown a heat exchanger 164 comprising a primary circuit 164a and a secondary circuit 164b and a pump device 164c. An electrolyte solution or pure water is conducted through line 189 through heat exchanger primary circuit 164a and a second heater 165, for example an electric heater controlled by a temperature sensor 169.

A first bag 152a comprising a heat sensitive first component such as glucose is connected through a connector 192a to a three-way valve 157a. The first component passes from the three-way valve 157a to a pump 161a and further to a mixing point 167a, in which the first component is heated to about 141° C. by mixture with a heated electrolyte component, having a temperature sufficient for promoting such heating by mixing, the temperature being for example 155° C. The mixing temperature is controlled by a temperature sensor 169a, which operates a throttle valve 193a arranged before the mixing point 167a. By throttling the valve 193a, a sufficient flow rate for obtaining said temperature is adjusted.

A second bag 152b comprising a heat sensitive second component, such an amino acids, is connected through a connector 192b to a three-way valves 157b. The second component passes from the three-way valve 157b to a pump 161b and further to a mixing point 167b, in which the second component is heated to about 141° C. by mixture with a heated electrolyte component, having a temperature sufficient for promoting ouch heating by mixing, the temperature being for example 155° C. The mixing temperature is controlled by a temperature sensor 169b, which operates a throttle valve 193b arranged before the mixing point 167b. By throttling the valve 193b, a sufficient flow rate for obtaining said temperature is adjusted.

The two heat sensitive components heated to sterilizing temperature by mixture with the electrolyte component are handled in parallel in two separate lines, 194a and 194b, which pass in parallel through the residence device 168, the pre-cooler 191, if present, and to heat exchanger secondary circuit 164b. After cooling in the heat exchanger, the two fluids are mixed in a Y-connector 195 before entering, the restriction device 70, see FIG. 2. The bags, 152a and 152b, are weighed and when a sufficient amount of fluid has been taken out from each bag, valve 157a and/or valve 157b are switched to stop the flow of first and/or second components from bags, 152a and 152b, respectively.

During sterilization, the three-way valves 157a and 157b are connected according to the broken lines in FIG. 3, in order to pass fluid, by means of pumps, 161a and 161b, in the fluid lines to and from the three-way valves, 157a and 157b, through lines 190a and 190b.

It is realized that more than two heat sensitive components may be handled in parallel by adding further bags 152 and further lines 194. Of course, the same procedure may be adopted for components which are less heat sensitive, to obtain a simple system, whereby the electrolyte component may be replaced with pure water, and thus, the electrolytes may be added one by one or several at a time.

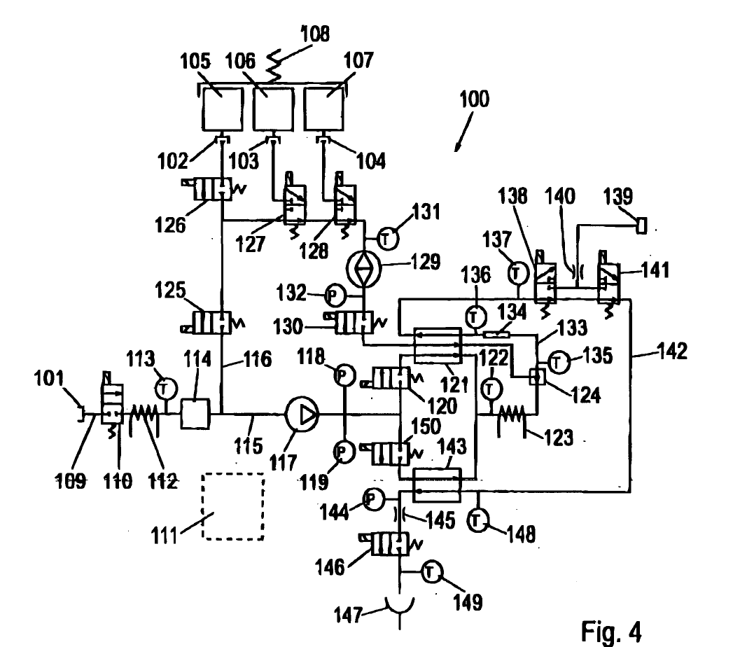

A further alternative embodiment of the present invention is shown in FIG. 4. From the left, the device 100 comprises a connector 101 for connection to a source of pure water, such as an RO-unit (not shown). The device further comprises three concentrate connectors 102, 103 and 104, which may be integrated into a single connector device. Each of connectors 102, 103 and 104 connects to a vessel or bag comprising a concentrate, such as a first bag 105 comprising a concentrated bicarbonate solution, a second bag 106 comprising electrolytes, such as sodium chloride, magnesium chloride, calcium chloride, and sodium lactate, at a predetermined pH, and a third bag 107 comprising glucose at a concentration of 50%. Of course, the bags include the components necessary for the final solution as discussed in more detail below. The components are divided into separate bags because they cannot be stored together or they cannot be sterilized together, or for other reasons.

Alternatively, one or more of the vessels or bags, 105, 106, 107, may comprise a powder instead of a solution in which case appropriate dissolution means may be provided.

Conveniently, the bags, 105, 106 and 107, are combined into a single assembly. The combined assembly of bags is attached to a weighing device 108, so that the weight of the assembly is monitored. The connectors, 102, 103 and 104, are attached to the ends of flexible tubes of PVC or other suitable pliable material, so that the connectors and tubes do not significantly influence the weight of the assembly.

The RO inlet connector 101 is connected to a line system including a first inlet line 109. Inlet line 109 is provided with an inlet valve 110, to isolate the device 100, if required. Inlet valve 110 is normally closed, but is opened upon activation by a control device 111 shown by broken lines. The control device may be a computer or microprocessor or any other control device. Normally, it is the control computer of the complete device.

Inlet line 109 further comprises a heater 112 and a temperature sensor 113, which operate together to adjust the temperature of incoming pure water to a predetermined temperature of, e.g., 25° C., in order, to make the device independent of incoming water temperature.

Inlet line 109 further comprises a flow meter 114 for measuring the complete inlet flow through inlet connector 101, for a purpose to be described later.

Downstream of flow meter 114, inlet line 109 is divided into water line 115 and concentrate line 116. Water line 115 comprises a first pump 117 for increasing the pressure of the water in water line 115 downstream of the pump to a pressure of about 2 to 6 Bar absolute pressure. The pressure is measured by a first pressure sensor 118 and monitored by a second pressure sensor 119. The first pressure sensor 119 is connected to the control system of computer 111, while the second pressure sensor 119 is connected to a parallel supervising system for ensuring the safety of the system. Several of the sensors are duplicated in this manner to provide independent data to the supervisory system or processor, even if not explicitly indicated in the drawings.

Water line 115 further comprises a valve 120 and a primary circuit of a heat exchanger 121. In the heat exchanger, the water in water line 115 is heated from about 25° C. to about 131° C. in heat exchanger 121, at a flow of about 120 ml/min. The temperature of the heated water is monitored by temperature sensor 122. Finally, water line 115 comprises a second heater 123, for heating the water to a still higher temperature, such as about 145° C. The hot water is delivered to a mixing point 124.

In concentrate line 116, the is a valve 125 for connecting the normally closed concentrate line 116 to water line 115. Further downstream, concentrate line 116 comprises three concentrate valves, 126, 127 and 128, and a reversible second pump 129. The second pump 129 is arranged to withdraw concentrate solutions or fluids from any one of concentrate bags, 105, 106 or 107, depending on the positions of valves, 126, 127 and 128. The second pump 129 further increases the pressure of the fluid in concentrate line 116 to a pressure of about 2 to 6 Bar absolute pressure.

Downstream of second pump 129 is arranged a valve 130, and therefrom, the concentrate fluid is delivered to a second primary circuit of heat exchanger 121 in order to preheat the concentrate solution from, e.g., room temperature to about 131° C. From heat exchanger 121, the concentrate solution is delivered to mixing point 124.

Upstream of the second pump 129 is arranged a temperature sensor 131 for measuring the temperature of the incoming concentrate fluid, and downstream of the second pump is arranged a pressure sensor 132 for measuring that sufficient pressure has been obtained. As indicated before, these sensors may be duplicated for supervisory purposes.

In mixing point 124, the two fluid lines, 115 and 116, are joined so that the heated water in line 115 is mixed with preheated concentrate in line 116 and the mixture is transported in mixed fluid line 133. Mixed fluid line 133 comprises a residence device 134, normally being a length of tube of a length to produce a predetermined residence time at a predetermined rate of flow to effect sterilization of the fluid in the residence device 134. The residence device 134 is preceded by a temperature sensor 135 and followed by a temperature sensor 136. These temperature sensors control the heater 123 to ensure that sterilizing conditions are obtained in the residence device 134, such as a minimum temperature of 141° C. for 12 seconds.

From the residence device 13, the sterilized and mixed fluid is passed to the secondary circuit of heat exchanger 121, at a temperature of approximately 141° C. The sterilized fluid is rapidly cooled to about 37° C.

Downstream of the heat exchanger, mixed fluid line 133 comprises sterilized fluid at a temperature suitable to be delivered to a patient or a storage bag. The temperature is monitored by a temperature sensor 137. Finally, a valve 138 directs, when activated, the fluid to an outlet connector 139, through a restrictor device 140, for lowering the pressure to atmospheric pressure.

The restrictor device may be a small hole in a piece of metal, the hole being dimensioned to reduce the pressure from about 6 Bar to about 1 Bar at the desired flow rate of, for example, 140 ml/min. An alternative design would be to use a controllable throttle valve, which is controlled by the processor in dependence of pressure sensor readings. A third alternative would be to use a throttle device or the pressure relief type, which adjust the differential pressure over the throttle device to a predetermined pressure drop of, for example, 5 Bar. A fourth alternative would be to use a throttle device controlled to deliver fluid at an output pressure of no more than a predetermined safe pressure of, for example, 1.25 Bar, in which case the pumps are operated to ensure that the pressure before the throttle device is sufficiently high, for example 6 Bar.

It is noted that the on-line autoclave as described is always operated at a predetermined minimal flow rate of not less than a predetermined flow rate, for example 120 ml/min, in order to ensure that the autoclave is maintained sterile. As soon as the flow rate drops below said predetermined minimum flow rate, the sterility conditions may be hampered or the autoclave may not be controlled to operate at proper temperatures. The autoclave may be designed to operate at different flow rates above said minimum flow rate. In order to always maintain a minimal flow rate, any excess fluid produced may be sacrificed to the waste.

If the mixed and sterilized fluid cannot be delivered out through the output connector 139, a valve 141 is activated to deliver the fluid to a waste receiver through a waste line 142. Waste line 142 further comprises a primary circuit of a second heat exchanger 143, a pressure sensor 144, a restrictor device 145 and a valve 146 until the fluid is delivered to the waste receiver 147. A temperature sensor 148 arranged upstream of heat exchanger 143 and another temperature sensor 149 arranged downstream of valve 146 are used to measure the temperatures of the waste fluid.

The device according to FIG. 4 may be operated in different modes. One mode of operation will be described below, namely sequential delivery of the components of the final fluid. It is, however, understood that the device may operate as described in connection with FIG. 2 as well.

In the sequential operation mode, water is first delivered in inlet line 109 at a constant rate of 120 ml/min from inlet connector 101, through flow meter 114, in which the flow rate is monitored, and through water line 115 and through first pump 117 to raise the pressure, so that the boiling temperature of the fluid is above the temperature anywhere in the circuit. If the maximum temperature is about 150° C., the pressure should be above 4.8 Bar or preferably about 6 Bar absolute pressure. The exact pressure is dependent on the adjustment and operation of restrictions device 140. The water further passes the mixing point 124 and enters the mixed fluid line 133 and reaches valve 138, which directs the flow to waste line 142, through valve 141 and further to the sump. The outlet connector 139 is connected to a recipient, normally a bag, such an a heater bag described below.

When all conditions are checked and the device delivers sterilized water, valve 138 is switched to direct the sterilized water to the outlet connector 139 through restrictor 140.

Substantially at the same time, or shortly thereafter, valve 127 in concentrate line 116 is opened and concentrate pump 129 is activated, with valve 130 in an open condition, to pump concentrate fluid from electrolyte bag 106, through heat exchanger 121 to mixing point 124. The concentrate pump 130 is operated to provide a flow rate of approximately 20 ml/min. At the same time, the weight of the concentrate assembly is monitored by weighing device 108. If the intention is to provide 1 liter of final solution and the concentrate fluid in bag 106 has a concentration of 1:40, the flow is continued for about 1 minute and 15 seconds, until the weighing device indicate that a volume of 25 ml has left the bag 106, whereby 25 ml is the amount required from concentrate bag in 1 liter of final fluid (1:40).

Then, valve 127 is switched off and valve 125 is opened for a short time, such as 15 seconds, to rinse the concentrate line 116.

For including the second concentrate, which may be glucose, bag 107 is connected to the concentrate pump by closing valve 125 and opening valve 128. If the glucose concentrate fluid has a concentration of 50%, the concentrate pump is driven 1 minute per percent concentration to be required in the final fluid at 20 ml/min. If 4% is required, which is the maximum contemplated for a PD fluid, the glucose concentrate is dosed in 4 minutes.

After this step, the concentrate line 116 is again rinsed with water, for example for 15 seconds.

Thereafter, the bicarbonate bag 105 is connected. The bicarbonate is normally stored at a concentration of about 1000 mmol/l. First, valve 125 is closed and valve 126 is opened so that concentrate pump 130 pumps bicarbonate fluid out of bag 105. The flow rate may be the same, 20 ml/min, and the mixing and sterilization of bicarbonate fluid is discontinued when the weighing device determines that the required quantity has been removed from bag 105. If the final solution should contain 15 mmol/l, tie concentrate pump is operated for 45 seconds to take 15 ml of concentrated bicarbonate solution out of bag 105.

Finally, the concentrate line is rinsed once again and water is delivered to the outlet connector, until the final volume of fluid has been delivered to the bag connected at the outlet connector, which is determined by flow meter 114 in combination with the weight losses measured by weighing device 108 and calculated into volumes by computer 111, taking into account the different densities of the concentrate fluids.

This final filling of water also means that the mix of fluid in the bag connected to the outlet connector is agitated and mixed thoroughly.

During the complete sterilization process described above, valves 138 and 141 are maintained in the same position directing all fluid to the outlet connector 139. Thus, all fluid produced is delivered to the receiver, thereby minimizing the time required for the preparation of the complete fluid.

Thus, it is also evident that all fluid exiting from the concentrate bags, 105, 106 and 107, is finally delivered out of connector 139, so that there is no waste of concentrate fluid.

In the example above, 1 liter of final solution has been prepared, but in PD it is more normal that 2 liters are generated each time, or any other volume as required by the user. It is understood that 2 liters may be produced by doubling the above-mentioned times or by repeating the production of 1 liter two times.

It is contemplated that the concentrate fluid bags may include concentrate fluid required for a final fluid volume of about 12 to 25 liters or more if required. Then, the above sequence is repeated for each batch of 2 liters to prepare.

In certain applications for PD, bicarbonate is not used, but lactate is used as the sole buffer. In that case, the third bag in the concentrate assembly is unnecessary, and only two bags may be used. In that case, valve 126 is always closed.

To, prepare one batch of 1 liter (1.5% glucose concentration), takes about 7 minutes and 45 seconds, supposing that the RO unit delivers pure water at 120 ml/min and 25 ml electrolytes, 15 ml bicarbonate and 30 ml glucose are used. Thus, the waiting time between each PD exchange of about 2 liters has to be more than 15.5 minutes. This might be limiting in some circumstances as appears from an explanation of the drain and fill phases of a PD treatment below.

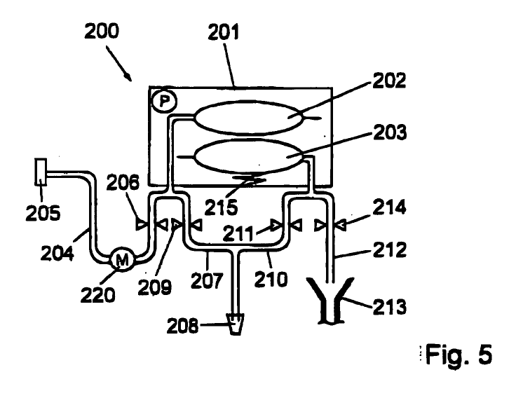

In FIG. 5 is schematically shown a PD cycler 200 intended to be used in the present invention. The PD cycler comprises a pressure chamber 201 enclosing a heater bag 202 and a waste bag 203. The heater bag 202 is connected to the outlet connector 139 of fluid sterilization device 100 of FIG. 4 for receiving a fresh sterilized fluid for introduction into heater bag 202. Heater bag 202 is connected with connector 139 through a first tube 204 ending with a connector 205 mating with connector 139 and comprising a valve 206. A second tube 207 connects heater bag 202 with a connector 208 to a patient (not shown) and the second tube 207 is controlled by a second valve 209. A third tube 210 connects the patient connector 208 to the drain bag 203 through a third valve 211. Finally, a fourth tube 212 connects drain bag 203 with a waste line 213 through a valve 214. Heater bag 202 and drain bag 203 rest on a pair of scales 215 which monitor the combined weight of the two bags.

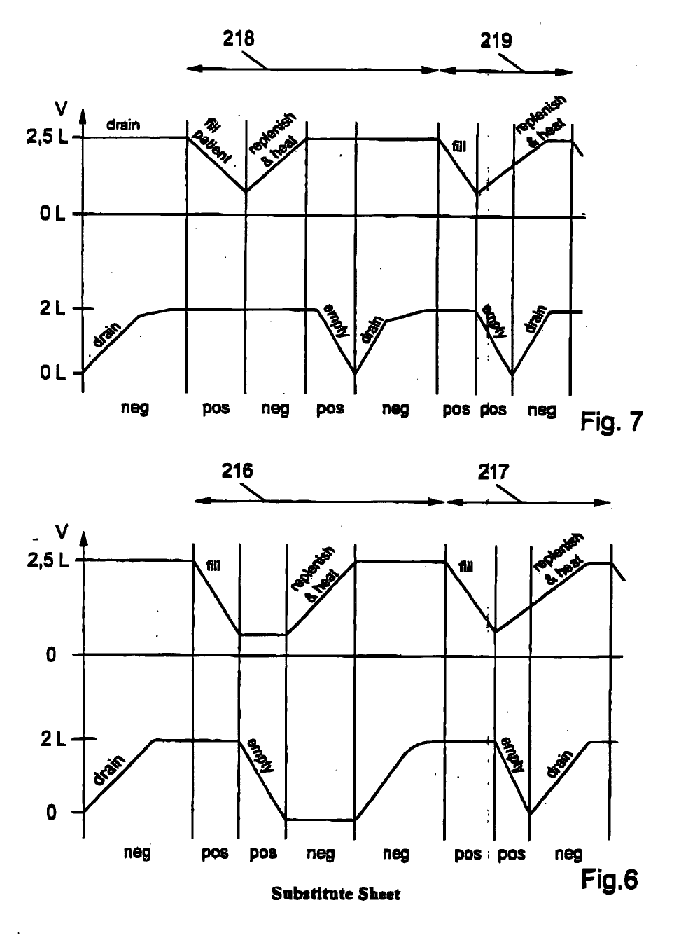

The operation of the PD cycler as schematically disclosed in FIG. 5, appears from the diagrams of FIGS. 6 or 7. The diagram indicates the fluid volumes of the heater bag and drain bag during the different phases.

After priming, which is more closely described below, the first phase of the treatment is a drain phase, at the start of which the heater bag is full of fluid, normally about 2.5 liters, and the drain bag is empty. The patient is connected and the third valve 211 is opened and a subpressure is exerted in pressure chamber 201. Fluid is withdrawn from the patient into drain bag 203 at a flow rate depending on the patient catheter and the subpressure, normally from about 150 to 300 ml/min. When the peritoneal cavity of the patient is almost empty, which may be indicated by a decrease of the drain flow as measured by the scales 215, the drain phase is terminated. The drain phase is normally about 7 to 10 minutes.

The second phase is a fill phase, in which the peritoneal cavity of the patient is filled with fresh fluid contained in heater bag 202. An overpressure is exerted in pressure chamber 201 and valve 209 is opened, while the other valves are closed. The fill flow rate depends on the patient and the overpressure and may be about 150 ml/min. The fill phase is normally about 10 to 15 minutes.

The third phase is an empty drain bag phase, in which an overpressure is exerted in the pressure chamber 201 and valve 214 is open. The fluid in the drain bag is directed to a waste line 213. The volumes are always monitored by the scales 215. The third phase may be about 2 minutes, since a high overpressure may be used and the flow restriction is minimal.

The fourth phase is heater bag replenishment phase with valve 206 open. In this case, normally a subpressure is exerted in the pressure chamber 201. Fluid is received from the sterilizing device 100 connected to connector 205 at a flow rate of about 120 ml/min. The fourth phase is normally about 15 to 17 minutes.

Thus, a complete cycle is about 34 to 44 minutes. During a night treatment of 8 hours, it is possible to exchange about 22 to 28 liters, in batches of 2 liters.

As shown in FIG. 7, the emptying phase and the replenishment phase may be interchanged.

If it is desired to increase the fluid volume further, the times in the different phases have to be shortened. It is noted that the heater bag fill time of about 15 to 17 minutes could be shortened by increasing the flow rate of fluid from sterilizer 100. However, increasing the flow rate means considerable cost increases

Instead, it is noted that the flow rate of the fluid delivered from sterilizer 100 is monitored by the sterilizer by flow meter 114 and weighing device 108. Thus, it is possible to replenish the heater bag during (part of) the drain cycle as shown in FIGS. 6 and 7. This is done by opening valve 211 while valve 209 is closed during the heater bag replenishment phase. If the drain phase is terminated before the heater bag is replenished, the patient fill phase cannot start until the heater bag replenishment is completed. However, it is no drawback to continue the drain phase longer, since that only results in some further fluid being drained, which normally is an advantage. Since the replenishment flow from the sterilizer is known, the PD cycler still has full control of the flows by using the reading from the scales and subtracting the replenishment flow obtained from the sterilizer. In this way, almost the complete drain phase can be saved in the cycle time, i.e., up to 10 minutes.

In FIG. 6, the normal cycle time is shown by arrow 216 while the shortened cycle time according to the present invention is shown by arrow 217. In FIG. 7, the normal cycle time is shown by arrow 218 while the reduced time according to the present invention is shown by arrow 219. In fact, the two cases of FIGS. 6 and 7 becomes the same according to the present invention, see arrows 217 and 219.

In FIG. 7, it is shown that the replenishment phase starts immediately after the fill phase. However, it is understood that it can start any time during the emptying phase or the following drain phase. However, by starting the replenishment phase as soon as possible, longer time is obtained for heating the replenishment fluid to 37 degrees Celsius.

In FIGS. 6 and 7, the pressure in the pressure chamber is indicated at the bottom by “neg” and “pos”, indicating a subpressure or an overpressure. Since the replenishment phase does not need a negative pressure, there is only one positive period and one negative period of pressure in a cycle, compared to two of each in the normal cycle of FIG. 7. This will result in a saving of the power required for the air pump in the cycler and a reduction of the sound level. The replenishment takes place by means of the volumetric pump and overpressure in the autoclave, possibly monitored by a flow meter, such as flow meter 220 shown in FIG. 5.

In this operation mode, it is still possible to maintain accurate control over the ultrafiltration, since the volume of fluid drained from the patient and the volume of fluid filled into the patient are under full control of the mass balance device 215.

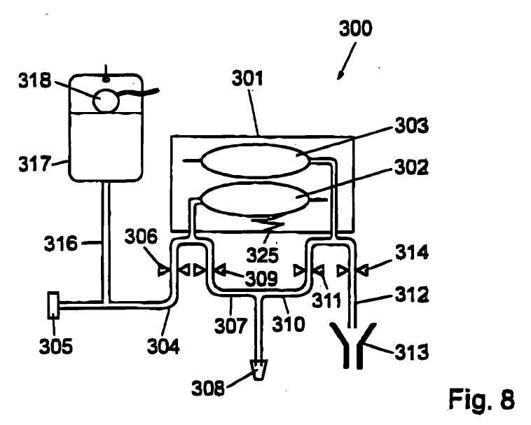

If the cycle time needs to be further shortened, that is possible by the addition of a storage, bag in the line set as indicated in FIG. 8 It is noted that the sterilizer has to direct the sterilized fluid to the waste 147 during the second phase filling the patient, when valve 206 is closed, as well as under the third phase emptying the drain bag.

In FIG. 8, the same components as in FIG. 5 have received the same reference numeral starting with 3 instead of 2. The inlet tube 304 is provided with a branch line 316 ending in a storage bag 317. When valve 306 is closed during the first, second and third phase, the sterilizer 100 delivers PD solution into storage bag 317 through tube 316. The heater bag 302 may then be replenished much faster from the storage bag 317 compared to the embodiment of FIG. 5. Thus, the heater bag replenishment phase may be seduced to 2 minutes or less. The efficiency of the complete device becomes dependent only on the cycler and its capacity to drain and fill the patient. The surplus time is merely 4 minutes, 2 minutes for emptying of the drain bag and 2 minutes for replenishment of the heater bag. The procedure has to be controlled if the sterilizer is operated in the sequential mode as described in connection with FIG. 4, since the filling of heater bag has to start only when the concentrations are correct in storage bag 317, i.e., after the completion of an entire fill cycle from the sterilizer.

The storage bag may also be used as an entry point for addition of medicaments or other additions, like insulin, antibiotic drugs, potassium chloride etc.

It is recognized that the PD solution produced according to the sterilizer in FIG. 4 will produce sterile bicarbonate fluid and enter it in the storage bag 317, and then produce sterile glucose solution and subsequently enter that in the storage bag 317. Since the glucose fluid has a low pH, some of the bicarbonate will react and form carbon dioxide, which may be released as a gas. Thus, storage bag 317 is provided with a valve and tube arrangement 318 to indicate when there is surplus gas in the storage bag 317 and expel it to the atmosphere. Another means for doing the same would be to include a sterile filter or hydrophobic filter at the top of storage bag 317. The gas may be expelled in a time interval when outlet valves 138 and 140 are opened (the position shown in FIG. 4) and pressure chamber 301 has an overpressure and valve 306 is open to exert an overpressure into storage bag 317 and expel gas therein.

In the above example indicated in connection with FIG. 4, the bicarbonate concentrate was sterilized at a concentration of about 140 mmol/liter(1000×20/140). However, there is a risk that carbon dioxide is formed during heat sterilization at such a concentration, and thus, the concentrate pump may be operated at a lower speed during sterilization of bicarbonate fluid.

In FIG. 4, the concentrate fluid is preheated to quite a high temperature. This is performed in an efficient heat exchanger 121 in which the heating fluid is the final sterilized fluid in the secondary circuit of the heat exchanger. Thus, the heat exchangers cannot have any point with higher temperature than the sterilizing temperature, and decomposition of the heat sensitive component is minimized. The further heating to the final sterilization temperature, i.e., from about 131° C. to about 141° C. takes place by the method of mixing with a fluid having a slightly higher temperature. Thus, the heat sensitive fluid component is never exposed to harsh conditions, such as hot points having excessively high temperatures, as may appear in an electric heater 123. Thus, favorable conditions for less formation of degradation products are obtained. The temperature difference between the primary and secondary circuits of the heat exchanger is about 10° C., which is possible to obtain without excessively long residence times in the heat exchanger.

In FIG. 4, there is a circuit not previously described for sterilizing the equipment before use. In water line 115, a parallel circuit to valve 120 and heat exchanger 121 is arranged comprising valve 150 and the primary circuit of heat exchanger 143. When heat disinfection of the complete sterilizer 100 is to be performed before a treatment, valve 120 is closed, valve 150 is opened and heater 123 is operated. The water passes from pump 117 through valve 150 to heat exchanger 143 and further to heater 123 to be heated to a temperature of, for example, 141° C. The hot water passes heat exchanger 121 but is not cooled appreciably since the primary circuit of exchanger 121 is disconnected and has no flow. The hot water after heat exchanger 121 passes through line 133 and through valves 138 and 141 to heat exchanger 143 to give off its heat to the water passing at the primary side thereof. Finally, the water is discharged to the waste through restrictor device 145, which lowers the pressure from about 2 to 6 Bar to atmospheric pressure.

Thus, the on-line autoclave is self-sterilized and is ready for producing PD fluids. The self-sterilizing step may be performed in about 30 minutes land is initiated under program control to happen shortly before the start of a PD treatment, which is scheduled in advance by a patient. When the self-sterilization process is ready, the machine awaits the arrival of the patient, which connects a disposable set, such as set 200 or 300 to the outlet connector 139. Then, the device produces a quantity of sterile treatment fluid into heater bag. However, before the patient is connected to connector 208, the tubes should be filled with fluid to displace the air therein. This is performed by attaching the connector 208 to a hook or attachment device on the cycler at approximately the same level as the heater bag. Then, valve 209 is opened to allow fluid to flow through tube 207 to patient connector 209. Then, the connector 208 is ready for connection to the patient.

It is appreciated that the priming procedure described above takes about 20 minutes since the heater bag must be filled with 2 liters of solution. If this time is too long for the patient to wait, it is possible to perform a partial fill of the heater bag with, for example, 5 dl solution produced in about 4 minutes, and use this volume of fluid to prime the tubes and displace the air. Then, the patient may connect himself to the connector 208 already after 4 minutes of priming and then go to bed, while the machine produces the first fill volume. It is noted that there is normally about 2 to 5 dl of solution left in the heater bag, in order to prevent complete emptying of the heater bag, because there is often some air or gas in the top of the heater bag, which should not be delivered to the patient. The first priming solution may be different from the treatment solution, for example comprising physiological sodium chloride.

It is recognized that the present invention for reducing the cycle time may be used with other sources of fresh fluid, such as supply bags as is conventional in APD. In this case, a pump and possibly a flow meter is added to perform the replenishment phase during the drain phase and/or the emptying phase, when the heater bag is not used by the cycler.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A method of peritoneal dialysis utilizing a pressure chamber including a first bag including an inlet for containing a fresh fluid for supply to a patient and a second bag for containing a spent fluid drained from a patient, and weighing means for weighing said first and second bags, said method comprising draining said spent fluid from said patient into said second bag by applying a reduced pressure to said second bag while weighing said first and second bags by said weighing means so as to control said draining of said spent fluid, and supplying said fresh fluid to said first bag at a predetermined replacement flow rate during said draining of said spent fluid from said patient into said second bag independently of said influence of said reduced pressure.

2. The method of claim 1 including applying said reduced pressure to both said first and second bags during said draining of said spent fluid from said patient into said second bag.

3. The method of claim 1 including supplying said fresh fluid to said first bag at said predetermined replenishment flow rate by means of a volumetric pump provided at said inlet of said first bag.

4. The method of claim 1 wherein said predetermined replenishment flow rate comprises a constant flow rate.

5. The method of claim 4 including supplying said fresh fluid to said first bag at said constant flow rate until a predetermined replacement volume of said fresh fluid has been supplied to said first bag.

6. The method of claim 1 including controlling said draining of said spent fluid from said patient into said second bag by means of said weighing means by correcting the amount of said spent fluid drained from said patient by the amount of said fresh fluid supplied to said first bag.

7. The method of claim 6 including terminating said draining of said spent fluid from said patient into said second bag when a predetermined volume of spent fluid has been drained into said second bag.

8. The method of claim 6 including terminating said draining of said spent fluid from said patient into said second bag when a predetermined time period has elapsed from the start of said draining of said spent fluid from said patient into said second bag.

9. The method of claim 6 including terminating said draining of said spent fluid from maid patient into said second bag when the rate of flow of said spent fluid into said second bag is below a predetermined flow rate, and including determining said rate of flow of said spent fluid into said second bag by means of said weighing means.

10. The method of claim 1 including filling said patient with said fresh fluid from said first bag and emptying said spent fluid from said second bag to a waste receiver by providing a positive pressure to said second bag, wherein said supplying of said fresh fluid to said first bag is carried out during said draining of said spent fluid from said patient into said second bag by pumping said fresh fluid at a predetermined flow rate independent of the influence of said negative pressure on said first bag.

11. The method of claim 10 including initiating said supply of said fresh fluid to said first bag during said emptying of said spent fluid from said second bag, and initiating said draining of said spent fluid from said patient after termination of said emptying of said spent fluid from said second bag.

12. The method of claim 10 wherein said filling of said patient with said fresh fluid is carried out under a positive pressure in said pressure chamber, said emptying of said spent fluid from said second bag is carried out under a positive pressure in said pressure chamber, and said supply of said fresh fluid to said first bag is carried out during either said emptying of said spent fluid from said second bag or said draining of said spent fluid under the control of a positive displacement pump irrespective of the pressure in said pressure chamber.

13. The method of claim 1 including filling said patient with said fresh fluid from said first bag and emptying said spent fluid from said second bag to a waste receiver by providing an increased pressure to said second bag, wherein said supply of said fresh fluid to said first bag and said draining of said spent fluid from said patient are carried out at least partially simultaneously.

14. The method of claim 13 including initiating said supplying of said fresh fluid to said first bag after termination of said filling of said patient with said fresh fluid from said first bag, and continuing said supplying of said fresh fluid to said first bag during said draining of said spent fluid from said patient in said second bag.

15. The method of claim 14 including continuing said supplying of said fresh fluid to said first bag during said emptying of said spent fluid from said second bag to said waste receiver.

16. The method of claim 1 including heating said fresh fluid to a temperature of about 37° C. during said supplying of said fresh fluid to said first bag.

17. The method of claim 16 including terminating said supplying of said fresh fluid to said first bag and initiating said filling of said patient with said fresh fluid from said first bag when said temperature of said fresh fluid in said first bag reaches about 37° C.
